(19) United States
(12) Patent Application Publication
(10) Pub. No.: US 2008/0040150 A1
(43) Pub. Date: Feb. 14, 2008

(54) METHODS AND APPARATUS FOR SEARCHING AND IDENTIFYING DISEASES

(76) Inventor: Philip M. Kao, Carlsbad, CA (US)

Correspondence Address: ANTOINETTE G. GIUGLIANO, P.C., 100 Cummings Center, Suite 342G, Beverly, MA, 01915, US

(21) Appl. No.: 11/463,318

(22) Filed: Aug. 9, 2006

(51) Int. Cl. G06Q 10/00; A61B 5/00

(52) U.S. Cl. 705/2; 600/300

(57) ABSTRACT

The present invention relates to methods, computer systems and computer apparatus for obtaining references that assist in identifying a disease or condition in an individual, wherein the reference has been described or made part of a database having medical data. The steps of the method include obtaining or identifying two or more symptoms, conditions, procedures, medication or any combination thereof, experienced or used by the individual; transcribing the symptoms, conditions, procedures, medication or combination thereof into standardized medical codes to thereby obtain transcribed codes; categorizing transcribed codes by medical specialty; searching at least one medical literature database using one of the transcribed codes to thereby obtain code-specific results, and repeating this step for each of the transcribed codes; cross-referencing the code-specific results for each transcribed code with code-specific results of all other transcribed codes in the same categorized medical specialty to obtain common references, and repeating this step for each specialty for which the codes have been categorized; and studying the references to determine references applicable to or those that assist in identifying the disease or condition.

The computer system or computer apparatus of the present invention includes a source of transcribed codes that comprises standardized medical codes translated from two or more symptoms, conditions, procedures, medication or any combination thereof experienced or used by the individual; a processor routine coupled to receive transcribed codes from the source, wherein the processor routine performs the above steps of categorizing transcribed codes by medical specialty; searching the medical literature database and cross-referencing the code-specific results for each transcribed code with code-specific results of all other transcribed codes in the same categorized medical specialty to obtain common references. The computer system or apparatus further includes an output device, coupled to the processor routine, for indicating common reference grouped by the number of occurrences of transcribed codes present in the reference.

### BACKGROUND OF THE INVENTION

Diagnosing an orphan disease or even a disease that is more prevalent can sometimes be difficult for many reasons. Often, individuals exhibit some, but not all, documented or known symptoms of a disease or condition. Additionally, orphan diseases are diseases that do not occur, or are difficult to identify, in a large percentage of the population. When diseases are described, physicians or researchers many times describe the same symptoms differently. Similarly, clinicians of a certain specialty sometimes tend to focus on symptoms, tests, and results common or related to their specialty and may not report tests results or findings that are common in another specialty. For example, an immunologist that focuses on autoimmune diseases may not test, report or describe a patient's kidney problem.

Consequently, a need exists for methods that obtain references that assist in identifying diseases or conditions that have been reported in medical databases. A further need exists to uniform the information relating to a patient's condition. Yet a further need exists to develop methods that can search and find relevant references even though medical databases have information about diseases that may be limited or incomplete.

### SUMMARY OF THE INVENTION

The present invention relates to methods for obtaining references that assist in identifying a disease or condition in an individual, wherein the reference has been described or made part of a database having medical data. The methods include obtaining or identifying two or more symptoms, conditions, procedures, medication or any combination thereof, experienced or used by the individual; and transcribing the symptoms, conditions, procedures, medication or combination thereof into standardized medical codes (e.g., International Classification of Diseases (ICD) codes, Current Procedural Terminology (CPT) codes, National Drug Codes (NDC) codes, or combination thereof) to thereby obtain transcribed codes. The methods further involve categorizing transcribed codes by medical specialty; searching at least one medical literature database using one of the transcribed codes to thereby obtain code-specific results, and repeating this step for each of the transcribed codes. The code-specific results of each transcribed code are cross-referenced with code-specific results of all other transcribed codes in the same categorized medical specialty to obtain common references. This step is repeated for each specialty for which the codes have been categorized. The references are then studied to determine references applicable to or those that assist in identifying the disease or condition. The methods further include studying a medical chart for the individual to obtain or identify two or more symptoms, conditions, procedures, medication or any combination thereof, exhibited or used by the individual. Once the cross-referencing occurs, a two-dimensional graph or representation that compares results for each transcribed code can be made.

Alternatively, the cross-referencing step, described above, can occur prior to the searching step in the methods of the present invention as follows. Such a method for obtaining references that assist in identifying a disease in an individual includes obtaining or identifying two or more symptoms, conditions, procedures, medication or any combination thereof, experienced or used by the individual; transcribing the symptoms, conditions, procedures, medication or any combination thereof into standardized medical codes to thereby obtain transcribed codes; categorizing transcribed codes by medical specialty; and then cross-referencing each transcribed code with all other transcribed codes in the same categorized medical specialty to thereby obtain cross-referenced code sets or pairs. Using the cross-referenced code sets, the method includes searching at least one medical literature database to thereby obtain cross-referenced, code-specific results, and repeating this step for each set or pairs of cross-referenced code. The references are then studied to determine references applicable to or those that assist in identifying the disease or condition.

In one aspect the present invention also includes obtaining one or more demographics (e.g., age, sex, race, species, geographic location or combination thereof) of the individual, and filtering out references having inconsistent demographics with the demographics of the individual. Filtering out results having inconsistent demographics involves excluding results that describe a demographic that is different from or at odds with the demographic obtained from the individual.

In an embodiment, the present invention, using the steps described herein, includes methods for identifying an orphan disease that has been described and made part of a database having medical information. In other embodiments, the present invention pertains to methods for assisting in determining a prognosis for an individual undergoing a procedure or in response to a type of therapy.

The present invention also includes methods for use in a computer system. The methods for use in a computer system include searching at least one medical literature database using one of the transcribed codes, as described herein, to thereby obtain code-specific results, and repeating this step for each of the transcribed codes. Such methods also include cross-referencing the code-specific results for each transcribed code with code-specific consistent results of all other transcribed codes in the same categorized medical specialty to obtain common references, and repeating this step for each specialty for which the codes have been categorized. The methods of the present invention further include providing an output that groups each common reference by the number of occurrences of transcribed codes present in the reference. The method further embodies obtaining one or more demographics of the individual and filtering out results having inconsistent demographics with the demographics of the individual.

The present invention also relates to a computer apparatus or system for obtaining references that assist in identifying a disease in an individual wherein the reference has been described or made part of a database having medical data. The apparatus has a source of transcribed codes that comprises standardized medical codes translated from two or more symptoms, conditions, procedures, medication or any combination thereof experienced or used by the individual; and a processor routine coupled to receive transcribed codes from the source. The processor routine performs the steps of categorizing transcribed codes by medical specialty; searching at least one medical literature database using one of the transcribed codes to thereby obtain code-specific results, and repeating this step for each of the transcribed codes. The process routine also cross-references the code-specific results for each transcribed code with code-specific results of all other transcribed codes in the same categorized medical specialty to obtain common references, and repeats this step for each specialty for which the codes have been categorized. The computer apparatus also has an output device, coupled to the processor routine, for indicating common reference grouped by the number of occurrences of transcribed codes present in the reference. The computer apparatus or system includes, in an embodiment, a filter, coupled to the source, for filtering out any results having inconsistent demographics with the demographics of the individual.

The present invention advantageously allows one to find relevant references that are likely to be helpful in diagnosing a disease or condition, or provide assistance in providing a prognosis of a patient in response to treatment or procedures. The present invention provides for uniformity of the input of a search, and a systematic methodology for reducing the number of irrelevant references.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a block diagram providing an overview of the process of an embodiment of the present invention.

FIG. 2 is a block diagram of a computer process employing the search methodology of the present invention.

### DETAILED DESCRIPTION OF THE INVENTION

A description of preferred embodiments of the invention follows.

The present invention relates to methods and systems for ascertaining or obtaining references that assist in identifying a disease (e.g., an orphan disease) in an individual. The references include those that have been made part of a database having medical information. Searching for references that are relevant to a specific individual's condition can be a difficult task, especially if the individual does not manifest several of the classic symptoms, or when not all symptoms are reported. Searching is also often difficult when the condition itself is not well documented. Searches for documented cases of related or similar conditions often result in numerous hits (citations and/or abstracts of references) that are irrelevant, or too few hits to be helpful. This occurs many times because a clinician or researcher's input or search strategy is flawed.

The present invention pertains to methods that result in a reasonable number of hits that are relevant or more likely to be relevant to the individual's specific condition, as compared references found using other search strategies. As such, references that assist in identifying a disease in an individual are those references that describe or likely describe one or more symptoms of at least one condition exhibited or manifested by the individual being studied. The method of the present invention, in an embodiment, reduces the number of irrelevant hits (e.g., hits describing unrelated symptoms or symptoms not related to the patient's condition) by at least about 50% (e.g., about 60%, 70%, 80%, 90%, or 95%).

The databases searched are those having medical data and/or information. Such databases can be medical databases, or non-medical databases having medical information. Both are referred to herein as “medical literature databases” or “medical database.” The databases include, e.g., databases having abstracts, keywords and/or full text information of medical journals, trade journals, patent information, medical presentations, case histories, medical seminar information, and any other medically related information.

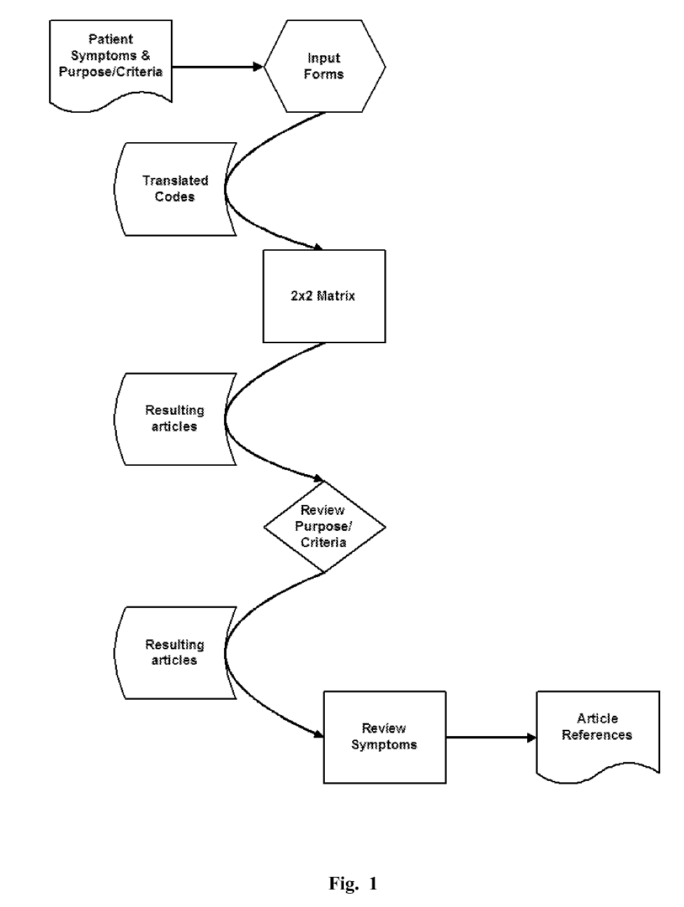

The present invention includes a number of steps. Referring to FIG. 1, a step of the present invention involves obtaining two or more (e.g., about two, three, four, five, six, seven, eight, nine or ten) symptoms, conditions, procedures performed, prescribed medications, or combination thereof exhibited or used by the individual. This step involves studying the entire chart of the patient/individual being studied, and is preferably performed by an individual (e.g., a scientist, a nurse, a doctor, a researcher, etc.) who understands a medical chart. In an embodiment, the chart includes medical information from all medical disciplines and specialties present. For example, the chart of a patient with an undiagnosed illness or condition will often include laboratory results, X-ray or imaging results, patient histories, symptoms exhibited, demographic information, medications, procedures performed, surgeries, etc. By studying an entire patient's chart including all information received by all medical specialists seen by the patient, more accurate information can be used to form the basis for the search.

Once the symptoms, conditions, procedures performed and/or prescribed drugs are extracted from the patient's chart, the present invention further includes transcribing them into a standardized medical code description and categorized by medical specialty or discipline, as further described herein and shown in FIG. 1. Transcribing relevant information is referred to herein to assigning a standardized description to the information. The advantage of the transcription step is to standardize the terminology utilized in the search. For example, an individual exhibiting hives can be referred to in a number of ways including rash, redness of the skin, allergic reaction of the skin, etc. However, when transcribing the symptom using a universally recognized code system, ICD, the symptom is described as “Rash and other nonspecific skin eruption” (ICD-10 Code R21). When utilizing the steps of the present invention, the text of the standardized code description is used in conjunction with other steps of the methodology, such as categorization of the code descriptions within a medical specialty. The information, when transcribed, is coded into the code description and referred to herein as a symptom code, condition code, procedure code, or medication code, collectively as a “transcribed code.”

Examples of standardized code systems include International Classification of Diseases (ICD) codes, Current Procedural Terminology (CPT) codes, National Drug Codes (NDC) codes, or combination thereof for symptoms, conditions, procedures, prescribed medications. The present invention includes using these standardized code systems, updates made to these code systems, and any other recognized code system now known or later developed. The methods of the present invention can be repeated using other types of code systems, which can depend on the type of condition and/or subject being studied. For example, the present invention, when applied to a medical condition in a human subject can utilize a medical standardized code system. In the case of a non-human subject, in addition to performing the invention using a medical code system the steps can be performed using a veterinarian code system. In yet another example, a human subject applied to an oral condition can include a step of transcribing the symptoms according a medical code system, a dental code system, or preferably the steps are repeated for both. Once the symptoms, conditions, procedures and/or prescribed drugs are transcribed, the codes are referred to herein as “transcribed codes.”

The transcribed codes, in an embodiment of the methodology of the present invention, include categorizing the codes by medical specialty or discipline. Often when patients' conditions are reported, they are done so by a specialist of a particular medical specialty. As such, the specialist many times report symptoms or focus on symptoms that are related to or common in that particular specialty. The categorizing of the codes by medical specialty increases the number of relevant hits. For example, a patent can have a medical chart that describes unknown autoimmune disease, unknown immunodeficiency, lymphopenia and prescribed prednisone which are related or classified as being common in immunological specialty of medicine. The same patient can also exhibit symptoms such as eosinophilic folliculitis, renal tublar acidosis, peripheral eosinophilia, autoimmune interstitial nephritis, and prescribed imuran, which all relate to nephrology as a medical specialty. There are numerous medical specialities and some include cardiology, immunology, nephrology, pediatrics, geriatrics, endocrinology, epidemiology, infectious disease, genetics, obstetrics, gynecology, psychiatry, neurology, orthopedics, oncology, radiology, etc. Medical specialities that are encompassed by the invention are those that are known in the art or later developed or recognized.

The present invention further includes searching one or more databases, as described herein, using the transcribed symptom, condition, procedure, and/or prescribed drug code, and repeating the search for each transcribed code to obtain code-specific results. The code-specific results for each transcribed code are cross-referenced with the results of the all other transcribed code in the same categorized medical specialty. In the example above, each transcribed code (autoimmune disease, unknown immunodeficiency, lymphonia, prescribed prednisone, eosinophilic folliculitis, renal tublar acidosis, peripheral eosinophilia, autoimmune interstitial nephritis, and prescribed imuran) would be individually searched, and the results for transcribed codes: autoimmune disease, unknown immunodeficiency, lymphopenia will be cross-referenced because they are in the same specialty, namely, immunology, as follows:

autoimmune disease—10,204 hits
unknown immunodeficiency—3,357 hits
lymphopenia—4,372 hits
prednisone—34,732 hits

TABLE 1
A two-dimensional representation of the cross-referenced hits for
symptom codes for a medical specialty.

| | AD | UI | Ly | Pr |
|---|---|---|---|---|
| AD | | 14 | 48 | 128 |
| UI | 14 | | 62 | 0 |
| Ly | 48 | 62 | | 89 |
| Pr | 128 | 0 | 89 | |

AD = autoimmune disease
UI = unknown immunodeficiency
Ly = lymphopenia
Pr = prednisone

As evidence by the cross-referencing step within a medical specialty, the number of hits is significantly reduced to a manageable number, and simultaneously removes irrelevant hits and leaves relevant hits, to thereby result in more meaningful results.

Table 1 is a two-dimensional representation of the crossed-referenced results. The cross-referencing step can be done manually or by computer software. One can graph, create a table, or otherwise make a representation of the number of cross-referenced hits.

Similarly, the hits from transcribed codes, eosinophilic folliculitis, renal tublar acidosis, peripheral eosinophilia, autoimmune interstitial nephritis, and prescribed imuran, can also be cross-referenced since they too are in the same medical discipline, namely nephrology. The process, as shown above, reduces the number of irrelevant or unrelated hits.

Alternatively, the transcribed codes can be cross-referenced and then searched together as a set or pair. For example, transcribed codes can be searched together, as a transcribed code set, in a database and the hits obtained. The cross-referencing can occur before or after the search to achieve the same set of relevant hits.

In addition to search using the transcribed codes, the present invention also includes narrowing the search results using patient demographics. Examples of demographics that can be included are age, sex, race, species, geographic location (e.g., current location or location where the patient lived for a considerable amount of time), or combination thereof. The hits can be categorized according to a desired demographics, and then excluded if the particular reference describes a patient study having an inconsistent demographic. An inconsistent demographic is one that is at odds with a demographic of the individual being studied. The use of filtering out references based on a desired demographic not only depends on the condition being presented, but also on the type of results desired. If the individual being studied requests references that describe only human results, then animal studies or in vitro experiments can be excluded by filtering out any reference with a non-human subject. Generally, results describing treatment of non-human subjects are considered to be experimental and too remote to be useful to a human being in the near future. As such, use of filtering out cross-referenced hits using a species demographic is useful if the individual being studied desires only treatments that are currently in use in humans or close to approval for such use.

In yet another example, filtering out references can be done according to age. After obtaining cross-referenced results for a 3-year old individual being studied, references that describe a 60-year old patient, if desired, can be excluded. However, in the case where age is not described at all, then the reference remains included in the hit list. The filtering of demographics can be done manually by a person studying the reference and, in certain instances, can also be automated. In the example above involving a 3-year old individual being studied, references referring to “geriatric” patients or studies could be eliminated manually by a person, or by a computer that can search the full text, abstract and/or keywords of the reference for the term “geriatric.”

Demographics used can be general or specific in nature. For example, in some instances, geographic location can be the country in which a person lives, or a specific city or town. The type and specific of the demographic used to narrow a search will depend on the nature of the condition being studied as well as the number of hits obtained from the methodology. For example, when conducting a search using the steps of the present invention, the hits obtained include references in which individual became sick from a contaminated water supply. Further limiting the hits by excluding references of individuals not from specific towns or counties of the individual being studied can be done in accordance with the present invention. In other instances, a geographic demographic limitation that is more broad is more appropriate and covers the country in which the individual being studied has lived or traveled to, or language-speaking countries of the individual.

In a preferred embodiment, the reference having demographic information is reviewed by the individual conducting the search and ruled out if the demographic information is inconsistent with the demographic information of individual being studied. In one aspect, the references are filtered out based on demographic information after the search results for the transcribed codes, they are cross-referenced within their medical specialty, as described herein. Filtering references refers to removing references that do not match the desired criteria (e.g., a reported age having more than a 50-year difference)

The present invention also includes a step of studying the references or abstract thereof to determine whether the references in the hit list are likely helpful to assist one (e.g., an expert such as a doctor, researcher, nurse, etc.) in identifying the disease or condition. The references are studied to determine the relevance to the condition of the person being studied. The references, in one aspect, are then grouped or weighed according to relevance or likelihood to be applicable to condition being studied. The individual reviewing the references is one that is qualified to understand the references. For example, such an individual (e.g., doctor, veterinarian, dentist, researcher, nurse, physician's assistant) is one that has training and/or education in the field, or one that can understand the references or abstracts thereof. In one embodiment, analyzing the references includes studying the list of symptoms cited by the author of the reference. Often authors list symptoms in order of significance, e.g., listing the most important one first, and so on. Relevance, in an embodiment, can be attributed by studying this list and assigning a greater weight to those references having the symptom codes higher on the list, and a lessor weight to those references having the symptom codes lower on the list or not listed at all (e.g., discussed elsewhere in the paper). In another embodiment, the references are weighted using a computer software program that scans the references and determines the occurrence of the transcribed codes in the abstract, full text or keyword list, and weighs the references accordingly. In an example, the more occurrences of one or combination of the transcribed codes provide greater weight than a reference having fewer occurrences. More weight, in an embodiment, can be given to combinations of more than two transcribed codes. The relevance can be determined using a suitable mathematical weighted average formula.

There are several applications of the present invention. The present invention, in an embodiment, can be used to assist one in identifying an uncommon condition or disease, such as an orphan disease. The National Institutes of Health has defined “orphan diseases” as a disease that affects 1 in every 200,000 people. With the US population at nearly 300 million, that translates to approximately 1500 unique orphan diseases. On the other hand, the present invention can be used to search symptoms associated with common or prevalent diseases or conditions to obtain references to assist in determining their potential outcome for a specific procedure or reaction to a particular type of medication (e.g., a method of determining references that assist in determining the prognosis of a patient by performing the steps described herein). For example, an eye surgeon wanting to research a specific condition of one of his patients before conducting a specific procedure can search for the symptoms (e.g., size of pupil, depth of cornea) that have been transcribed along with the procedure code, cross-referenced in the medical specialty to find relevant references. Similarly, the methods described herein can be used to determine references that assist in determining the prognosis of a patient in response to a specific treatment or procedure. The methods of the present invention can be used to conduct targeted research in the areas of medicine, dentistry and veterinarian medicine.

Portions or several of the steps described herein can be automated. Accordingly, the present invention includes a computer system, using the transcribed codes along with the associated medical specialty from the patients chart as input, searches one or more medical literature databases with a search engine or routine that compares the phrase of the transcribed code with the abstract, key words or full text of each reference in the database. The software can then cross reference the transcribed codes and provide an output or representation showing the results of the cross-reference and/or a representation that groups each common reference by the number of occurrences of the transcribed codes or combination of transcribed codes present in the reference.

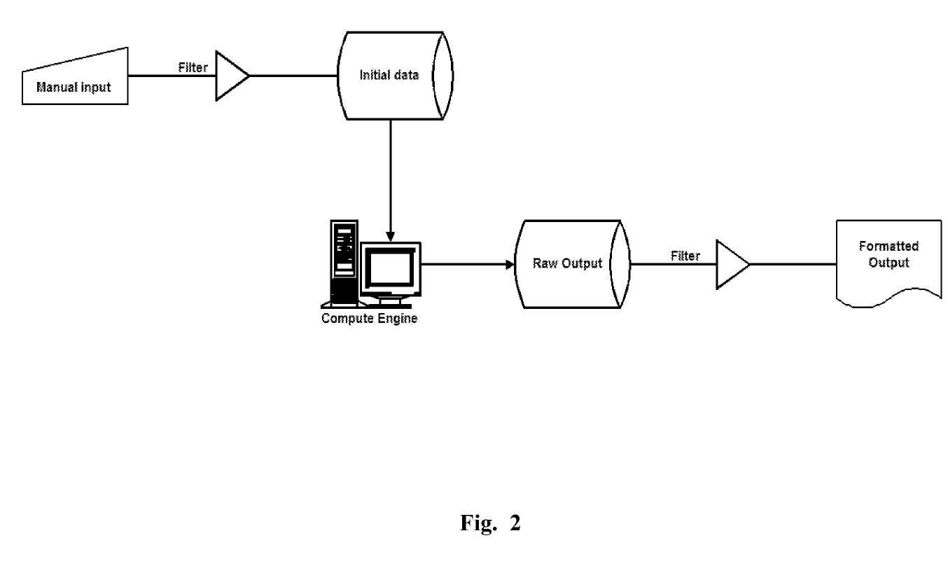

Referring to FIG. 2, a computer system embodying the steps of the methods described herein is an embodiment of the present invention. The computer system includes the manual input of data (e.g. transcribed codes and/or categories of medical specialties). The data can be filtered by reformatting the inputted data or abbreviate certain fields within the data. Filtering can be done with or without a computer engines. A computer engine manipulates the initial data, scans and/or searches the terms of the input data against the various medical databases available. The computer engine provides the raw output that can also be filtered a second time, with or without a computer engine. The data can be processed and formatted for output for either memory storage or display through an I/O device, e.g., a work-station display monitor, a printer, and the like. I/O processing (e.g., formatting) of the content is provided using techniques common in the art. The computer system according to the invention is useful in applications including, but not limited to, determining references that assist in determining a disease, a condition, response to therapy or procedure, as described herein.

Receiving the transcribed codes and associated medical specialty data refers to delivering data, which may or may not be pre-processed (e.g., resealed, filtered, and/or normalized), to the computer engine that searches the database and cross-references in the medical specialty. A processor routine refers to a set of commands that carry out a specified function. Once the computer engine performs the search, cross-referencing, and assigns weight to the references, then an output is provided which indicates the cross-referenced results and/or the hits according to their assigned weights. Providing an output refers to providing the datapoints to an output (I/O) device.

The references are then studied by a person that can understand the medical literature found by the search. The references are analyzed and weighted according to relevance and/or applicability.

### Exemplification

Four different search engines were used to perform the methods described below: medsite (www.medsite.com), emedicine (www.emedicine.com), melvyl (www.melvyl.com), and entrez (www.pubmed.gov).

The following is an overview of protocol that was employed, in accordance with FIG. 1.

Step 1. The medical professional inputted the patient symptoms and searched purpose/criteria into the computer system using the pre-process forms. These forms may be customized to fit specific needs.

### Step 2. The symptoms were translated into Codes based on the appropriate terminology and entered into the database.

### Step 3. The Codes were categorized by discipline and ranked by importance, based on the initial input order.

### Step 4. The Codes were input into a 2×2 matrix per discipline.

Step 5. The literature was searched using the 2×2 matrix. The results were tabulated.
Step 6. The resulting searches were scanned against the original input purpose/criteria. Articles which fail the purpose/criteria were moved to the bottom of the results list.

### Step 7. The remaining articles were scanned against the original patient symptoms and ranked by order of relevance.

### Step 8. The resulting article references were returned to the requesting medical professional.

The following description of a single patient was acquired from direct medical records and with the consent of the patient. The patient's identity has been removed.

The patient is described in direct medical records as:

### Oct. 12, 1992—eosinophilic pustular folliculitis

### Apr. 1, 1993—scaly lesions on head

### Apr. 21, 1993-8 months old, lethargic

Using the following keyword searches, the standard search engines returned the following citations:
eosinophilic pustular folliculitis

www.medsite.com returned 138 hits

www.emedicine.com returned 25 hits

www.melvyl.cdlib.org returned 20 hits on “eosinophilic”, 2 hits on “pustular”, 6 hits on “folliculitis”
but zero hits on all three words

entrez returned 128 hits(www.ncbi.nlm.nih.gov) returned 138 hits

scaly lesions

www.medsite.com returned 1 hit

www.emedicine.com returned 100+hits

www.melvyl.cdlib.org returned 6 hit on “scaly”, 908 hits on “lesions”
but zero hits on both words

entrez returned 128 hitsreturned 202 hits

lethargy

www.medsite.com returned 18865 hits

www.emedicine.com returned 100 hits

www.melvyl.cdlib.org returned 27 hits

entrez returned 128 hitsreturned 18865 hits

Using this same patient description, but transcribing the information first, yielded the following description:
infant eosinophilic pustular folliculitis

www.medsite.com returned 23 hits

www.emedicine.com returned 11 hits

www.melvyl.cdlib.org returned 0 hits

entrez returned 128 hitsreturned 23 hits

infantile eosinophilic pustular folliculitis

www.medsite.com returned 8 hits

www.emedicine.com returned 5 hits

www.melvyl.cdlib.org returned 0 hits

entrez returned 128 hitsreturned 8 hits

infantile eosinophilic scaly lesions

www.medsite.com returned 0 hits

www.emedicine.com returned 6 hits

www.melvyl.cdlib.org returned 0 hits

entrez returned 128 hitsreturned 0 hits

infantile lethargy

www.medsite.com returned 100 hits

www.emedicine.com returned 53 hits

www.melvyl.cdlib.org returned 0 hits

entrez returned 128 hitsreturned 100+hits

infantile eosinophilic scaly lesions lethargy

www.medsite.com returned 0 hits

www.emedicine.com returned 0 hits

www.melvyl.cdlib.org returned 0 hits

entrez returned 128 hitsreturned 0 hits

infantile lethargy scaly lesions

www.medsite.com returned 0 hits

www.emedicine.com returned 6 hits

www.melvyl.cdlib.org returned 0 hits

entrez returned 128 hitsreturned 0 hits

infantile eosinophilic lethargy

www.medsite.com returned 0 hits

www.emedicine.com returned 1 hit

www.melvyl.cdlib.org returned 0 hits

entrez returned 128 hitsreturned 0 hits

Using a different approach, the following letter describes the same patient. The purpose of the letter was to inform another set of doctors about the patient's current condition.

### May 4, 1995

### To Whom It May Concern:

<patient's name omitted> has a congenital immunodeficiency and requires treatment with intraveneous gammaglobulin. The use of gammaglobulin precludes active immunization with routine childhood vaccines. The gammaglobulin does protect against the illnesses in a fashion similar to vaccination.

### If I can provide further information, please contact me at <phone omitted>

### Sincerely,

### The same doctor described this patient in a case report

<patient's name omitted> is a 10 year old female with a long standing history of autoimmune disease and unknown immunodeficiency. Shortly after birth she was diagnosed with eosinophilic folliculitis and peripheral eosinophilia. This resolved with treatment. At the age of 8 months she was discovered to have renal tublar acidosis secondary to autoimmune interstitial nephritis with concurrent eosinophilic infiltration of her kidneys. At that time she was also noted to have lymphopenia with an absolute lymphocyte count of approximately 400 and a substantial peripheral eosinophilia. T cell subsets demonstrated low CD4+, CD8+, and CD3+ cells with diminished proliferation in response to PHA. B cells were low-normal, quantitative immunoglobulins were elevated with the exception of IgE which was normal. Her HIV test was negative and ADA and PNP levels were normal. She has been immunized to both tetanus toxoid and H.influenza and had made adequate specific antibodies to Tetanus. Her Hib response was initially low but following subsequent immunizations she was able to mount a specific polysaccharide response to Hib. In addition, she had isohemagglutinis mainfested by blood type B+.
Her renal disease was treated with prednisone and imuran. During a prednisone taper she was noted to have the onset of an erythematous, scaling rash which began at the age of 19 months. Biopsy of the rash demonstrated lichenoid and atopic characteristics. Although she was treated with topical steroids and tracrolimus, the rash persisted. The intense pruritis was not alleviated by antihistamines. Involvement of scalp integument has resulted in diffuse alopecia. During a steroid taper at 6 years of age she developed Coomb's positive autoimmune hemolytic anemia requiring blood transfusions and increased steroid dosing. During her first presentation of anemia, she also had thrombocytopenia. Throughout this time her T cell subsets and absolute lymphocyte counts remained low as did her T cell proliferative response.

### Using both descriptions of this patient yielded the following citations. congenital immunodeficiency

www.medsite.com returned 2643 hits

www.emedicine.com returned 100+hits

www.melvyl.cdlib.org returned 722 hits on “congenital” and 372 hits on “immunodeficiency”
but zero hits on both words

entrez returned 128 hitsreturned 2643 hits

pediatric congenital immunodeficiency

www.medsite.com returned 202 hits

www.emedicine.com returned 100+hits

www.melvyl.cdlib.org returned 0 hits

entrez returned 128 hitsreturned 202 hits

infant congenital immunodeficiency

www.medsite.com returned 1346

www.emedicine.com returned 94 hits

www.melvyl.cdlib.org returned 0 hits

entrez returned 128 hitsreturned 1346 hits

infantile congential immunodeficiency

www.medsite.com returned 27 hits

www.emedicine.com returned 29 hits

www.melvyl.cdlib.org returned 0 hits

entrez returned 128 hitsreturned 27 hits

Typically, several keywords emerge when medically transcribing one specific discipline of a complicated patient symptoms. These keywords are only indicators of an underlying problem which may or may not have been previously described within the literature. However, finding all symptoms or keywords in a single article is unlikely. A 2×2 matrix was used to manipulate the keywords to extract pertinent information. This second procedure uniquely aligns the article searches into more meaningful results, thus provides references that assist in identifying the patient's underlying condition.

In the previous patient chart, several immunological and nephrological symptoms are described.

Immunological phrases:
autoimmune disease (condition)

www.medsite.com returned 10204 hits

www.emedicine.com returned 100+hits

www.melvyl.cdlib.org returned 213 hits

entrez returned 128 hitsreturned 10204 hits

unknown immunodeficiency (condition)

www.medsite.com returned 3357 hits

www.emedicine.com returned 100+ hits

www.melvyl.cdlib.org returned 6 hits

entrez returned 128 hitsreturned 3357 non-unique hits

lymphopenia (condition)

www.medsite.com returned 4372 hits

www.emedicine.com returned 84 hits

www.melvyl.cdlib.org returned 3 hits

entrez returned 128 hitsreturned 4372 hits

prednisone (drug)

www.medsite.com returned 34732 hits

www.emedicine.com returned 100+hits

www.melvyl.cdlib.org returned 19 hits

entrez returned 128 hitsreturned 34732 hits

Nephrological phrases:
eosinophilic folliculitis (condition)

www.medsite.com returned 60 hits

www.emedicine.com returned 29 hits

www.melvyl.cdlib.org returned 20 hits on eosinophilic, 6 hits on folliculitis,
but zero hits on the combined phrase

entrez returned 128 hitsreturned 60 hits

peripheral eosinophilia (condition)

www.medsite.com returned 535 hits

www.emedicine.com returned 100+hits

www.melvyl.cdlib.org returned 1 hit

entrez returned 128 hitsreturned 535 hits

renal tublar acidosis (condition)

www.medsite.com returned 0 hits

www.emedicine.com returned 0 exact matches, 100+non-unique hits

www.melvyl.cdlib.org returned 5050 hits on renal, 0 hits on tublar, 89 hits on acidosis

but zero hits on the combined phrase

entrez returned 128 hitsreturned 0 hits

autoimmune interstitial nephritis (condition)

www.medsite.com returned 27 hits

www.emedicine.com returned 81 hits

www.melvyl.cdlib.org returned 531 hits on autoimmune, 305 hits on interstitial, 228 hits on

nephritis, but zero hits on the combined phrase

entrez returned 128 hitsreturned 27 hits

imuran (drug)

www.medsite.com returned 11007 hits

www.emedicine.com returned 100+ hits

www.melvyl.cdlib.org returned 0 hits

entrez returned 128 hitsreturned 11007 hits

Now, one immunological term was cross-referenced with another, creating a 2×2 matrix of terms. This matrix is then searched using the four index engines listed above. The resulting searches dramatically eliminate the unnecessary articles hits from using a single term, and from using random keywords gleaned from the information itself.

TABLE 2
Immunological 2 × 2 matrix

| Immunological | | | | |
|---|---|---|---|---|
| Terms | AI | UI | Ly | Pr |
| AI | duplicate | Search #1 | Search #2 | Search #3 |
| | AI + AI | AI + UI | AI + Ly | AI + Pr |
| UI | duplicate to | duplicate | Search #4 | Search #5 |
| | search #1 | UI + UI | UI + Ly | UI + Pr |
| Ly | duplicate to | duplicate to | duplicate | Search #6 |
| | search #2 | Search #4 | Ly + Ly | |
| Pr | duplicate to | duplicate to | duplicate to | duplicate |
| | search #3 | Search #5 | Search #6 | Pr + Pr |

Key:
AI = autoimmune disease
UI = unknown immunodeficiency
Ly = lymphopenia
Pr = prednisone

Immunological 2×2 matrix results:
1. “autoimmune disease” and “unknown immunodeficiency”

www.medsite.com returned 14 hits

www.emedicine.com returned no exact match

www.melvyl.cdlib.org returned 0 hits

entrez returned 128 hitsreturned 14 hits

2. “autoimmune disease” and “lymphopenia”

www.medsite.com returned 48 hits

www.emedicine.com returned 36 hits

www.melvyl.cdlib.org returned 0 hits

entrez returned 128 hitsreturned 48 hits

3. “autoimmune disease” and “prednisone”

www.medsite.com returned 128 hits

www.emedicine.com returned 100+hits

www.melvyl.cdlib.org returned 0 hits

entrez returned 128 hitsreturned 128 hits

4. “unknown immunodeficiency” and “lymphopenia”

www.medsite.com returned 125 hits

www.emedicine.com returned 26 hits

www.melvyl.cdlib.org returned 0 hits

entrez returned 128 hitsreturned 62 non-unique hits

5. “unknown immunodeficiency” and “prednisone”

www.medsite.com returned 15 hits

www.emedicine.com returned 100+hits

www.melvyl.cdlib.org returned 0 hits

entrez returned 128 hitsreturned 15 hits

6. “lymphopenia” and “prednisone”

www.medsite.com returned 89 hits

www.emedicine.com returned 35 hits

www.melvyl.cdlib.org returned 0 hits

entrez returned 128 hitsreturned 89 hits

TABLE 3
Nephrological 2 × 2 matrix

| Nephrological | | | | | |
|---|---|---|---|---|---|
| Terms | EF | PE | RTA | AIN | Im |
| EF | duplicate | Search #7 | Search #8 | Search #9 | Search #10 |
| | EF + EF | EF + PE | EF + RTA | EF + AIN | EF + Im |
| PE | duplicate to | duplicate | Search #11 | Search #12 | Search #13 |
| | search #7 | PE + PE | PE + RTA | PE + AIN | PE + Im |
| RTA | duplicate to | duplicate to | duplicate | Search #14 | Search #15 |
| | search #8 | Search #11 | RTA + RTA | AIN + AIN | RTA + Im |
| AIN | duplicate to | duplicate to | duplicate to | duplicate | Search #16 |
| | search #9 | Search #12 | Search #14 | AIN + AIN | AIN + Im |
| Im | duplicate to | duplicate to | duplicate to | duplicate to | duplicate |
| | search #10 | search #13 | search #15 | search #16 | Im + Im |

Key:
EF = eosinophilic folliculitis
PE = peripheral eosinophilia
RTA = renal tubular acidosis
AIN = autoimmune interstitial nephritis
Im = Imuran

Nephrological 2×2 matrix results:
7. “eosinophilic folliculitis” and “peripheral eosinophilia”

www.medsite.com returned 0 hits

www.emedicine.com returned 10 non-unique hits

www.melvyl.cdlib.org returned 0 hits

entrez returned 128 hitsreturned 3 hits

8. “eosinophilic folliculitis” and “renal tublar acidosis”

www.medsite.com returned 0 hits

www.emedicine.com returned 4 non-unique hits

www.melvyl.cdlib.org returned 0 hits

entrez returned 128 hitsreturned 0 hits

9. “eosinophilic folliculitis” and “autoimmune interstitial nephritis”

www.medsite.com returned 0 hits

www.emedicine.com returned 2 non-unique hits

www.melvyl.cdlib.org returned 0 hits

entrez returned 128 hitsreturned 0 hits

10. “eosinophilic folliculitis” and “imuran”

www.medsite.com returned 0 hits

www.emedicine.com returned 1 non-unique hit

www.melvyl.cdlib.org returned 0 hits

entrez returned 128 hitsreturned 0 hits

11. “peripheral eosinophilia” and “renal tubular acidosis”

www.medsite.com returned 0 hits

www.emedicine.com returned 54 non-unique hits

www.melvyl.cdlib.org returned 0 hits

entrez returned 128 hitsreturned 0 hits

12. “peripheral eosinophilia” and “autoimmune interstitial nephritis”

www.medsite.com returned 0 hits

www.emedicine.com returned 9 non-unique hits

www.melvyl.cdlib.org returned 0 hits

entrez returned 128 hitsreturned 0 hits

13. “peripheral eosinophilia” and “imuran”

www.medsite.com returned 0 hits

www.emedicine.com returned 15 non-unique hits

www.melvyl.cdlib.org returned 0 hits

entrez returned 128 hitsreturned 0 hits

14. “renal tubular acidosis” and “autoimmune interstitial nephritis”

www.medsite.com returned 0 hits

www.emedicine.com returned 12 non-unique hits

www.melvyl.cdlib.org returned 0 hits

entrez returned 128 hitsreturned 0 hits

15. “renal tubular acidosis” and “imuran”

www.medsite.com returned 0 hits

www.emedicine.com returned 8 non-unique hits

www.melvyl.cdlib.org returned 0 hits

entrez returned 128 hitsreturned 0 hits

16. “autoimmune interstitial nephritis” and “imuran”

www.medsite.com returned 2 hits

www.emedicine.com returned 14 hits

www.melvyl.cdlib.org returned 0 hits

entrez returned 128 hitsreturned 0 hits

The resulting articles were then screened manually against the patient description for a more germaine article. This level of search was accomplished by reading the abstracts to determine the purpose of the article and the potential correlation with the patient situation. The timing of this step is important in the success of finding a good article source.

Alternatively, the next step is grouping the articles by adding another search term against the remaining articles. However, experience suggests that using three specific patient descriptions in keyword searches, typically results in zero hits. Therefore, it is not particularly fruitful to attempt a third round of boolean searches.

This process has pared down literally tens of thousands of articles of potential keyword matches, into a few hundred into a handful of articles that assist in determining the patients condition. The goal of this process is to take a clinical description of a patient and attempt to offer some evidence in literature of that same patient description.

The appendix contains the summaries of the articles pooled from the 2×2 matrix screening process. Each article abstract is scanned against the initial search criteria, not the matrix. The initial search criteria described an infant with both immunological and nephrological problems. The articles are weighed as follows:

### All abstracts are numbered against their corresponding 2×2 matrix results.

1a) is dismissed since it describes psoriasis.
1b) helpful, as it describes abnormal immune responding T-cell and immune response directing B-cell activities.
1c) helpful, as it describes general issues with immunodeficiencies. however it appears to focus on adult symptoms.
1d) is dismissed as this article appears to describe in vitro analysis.
1e) is dismissed as this article appears to describe binding sites and not human symptoms.
1f) is dismissed as this article appears to focus on retroviruses relating to Hepatitis C.
1 g) is dismissed as this article appears to be a study in how environmental conditions change immune responses.
1h) is dismissed as this article focuses on a 65-year-old male patient with diabetes.
1i) is dismissed as this article focuses on mice infected with retrovirus induced leukemia.
1j) helpful, but only as an overview discussion on superantigens.
1k) is dismissed as this article focuses on Mooren's ulcer.
1l) helpful, as the subject is a child with IgA deficiency.
2a) is dismissed as this article focuses on multiple sclerosis.
2b) is dismissed as this article focuses on transferability of immunized mice.
2c) helpful, if the problem involves Bim or Bcl-2 cell types
2d) helpful, for describing the relationship between lymphopenia and interleukin-2 to CD4+CD25+ cells.
2e) shows potentail if the problem involves hemophagocytic syndrome (uncontrolled activiation of the cellular immune system).
2f) is dismissed as this article focuses on dendritic cells.
2g) helpful, for describing T-cell functionality
2h) is dismissed since the patient description did not include gastro-intestinal symptoms.
2i) helpful, for IL-2 effects.
2j) is dismissed since it describes lupus related symptoms.
2k) is dismissed for describing diabetic symptoms.
2l) is dismissed for describing in vitro test results
2m) helpful, for describing T-cell effects on regulatory activity.
2n) helpful, for describing T-cell activities.
2o) helpful, for describing CD4+T-cells.
2p) is dismissed for describing focusing lan5 gene.
2q) helpful, for longer term information, but not for suggested solutions.
2r) is dismissed for being too theoretical for use. but could be good background for CD40.
2s) is dismissed for focusing on gastritis.
2t) helpful, for background information on CD4+T-cells.
2u) helpful, for background information on CD4+CD25+T-cells.
2v) is dismissed as the article focuses on diabetes.
2w) is dismissed as the article focuses on gastritis.
2y) is dismissed as the article discusses pulmonary silicosis (effects adults).
2z) is dismissed as the article discusses rheumatoid arthritis.
2aa) helpful, as it discusses both T cells and B cells.
2bb) is dismissed as the article focuses on Sjogren's syndrome (white blood cells attack moisture-producing glands).
2cc) is dismissed as the artciel focuses on Sjogren's syndrom (white blood cells attack moisture-producing glands).
2dd) helpful, as this article overviews apoptotic defects.
2ee) is dismissed as this article discusses the effects of releasing hormones into the immune system.
2ff) is dismissed as this article focuses on patients with myasthenia gravis (autoimmune attack of the muscle-nerve junction).
2gg) is dismissed as the article describes the treatment of pemphigus vulgaris (auto-immune blistering of the skin and mucous membranes).
2hh) is dismissed as the article focuses on bullous pemphigoid (blistering of the skin).
2ii) is dismissed as the article focuses on thymic carcinoma (typically effects adults) and myasthenia gravis (autoimmune attack of the muscle-nerve junction).
2jj) is dismissed as this article describes adult thymectomy (typicl treatment for myasthenia gravis).
2kk) is dismissed as this article describes the effects of space travel on the immune system. interesting though.
2ll) is dismissed as this article focuses on hypertension.
2mm) is dismissed as the article focuses on lissencephaly (brain disorder where whole or parts of the surface of the brain appear to be smooth).
2nn) is dismissed as this article relates to diabetes.
2oo) is dismissed as this article focuses on thyroid-based diseases.
2pp) helpful, if the condition can be attributed to a decrease in T cell lymphocytes.
2qq) helpful, as a broad description of immune-based regulatory responses.
2rr) helpful, if the condition relates to IgA deficiences.
2ss) helpful, as the article broadly describes immune-related deficiencies.
2tt) is dismissed as this article focuses on sjogren's syndrome (white cells attack moisture-producing glands).
2uu) is dismissed as this article focuses on lupus.
5a) helpful, as “pityriasis lichenoides” refers to scaly rash with the appearance of lichens.
5b) helpful, as this article uses prednisone to help cure lymphoma.
5c) is dismissed as the patient does not exhibit signs of Hodgkin's disease (cancer of the lymph nodes).
5d) is dismissed as this article uses gamma globulin and prednisone to treat myasthenia gravis.
5e) helpful, as this article observes chemical imbalances in an infant.
5f) is dismissed as this article refers to HIV-infected adults with dermatofibromas (cancerous skin growth).
5g) is dismissed as this article focuses on Epstein-Barr virus, a potential precursor to HIV.
5h) is dismissed as “Ofuji papuloerythroderma” is typically found in adult men and exhibit diffuse red papules.
5i) helpful, as still's disease effects children as a general inflamation.
5j) is dismissed as otosyphilis focuses on hearing loss and dizziness, neither of which was observed in the patient.
5k) is dismissed as the patient is not described as having an infectious esophagitis.
5l) is dismissed as this article describes treatment results of HIV-infected patients.
5m) is dismissed as this article discusses radiographic results using different materials (angioimmunoblastic lymphadenopathy).
5n) is dismissed as this article uses radiographic techniques to identify T-cell deficiencies.
5o) is dismissed as the article focuses on granulomatosis (gathering of macrophages) and hypogammaglobulinemia (lack of IgG).
7a) is dismissed as the eosiniphilic folliculitis was contracted after a bone marrow transplant.
7b) is dismissed as this article measures peripheral eosinophilia in HIV patients.
7c) helpful, as this article discusses treatment patterns of patients suffering from folliculitis.
10a) helpful, as this article discusses treatment and characterization of patients with various skin abnormalities (dermatosis).
12a) helpful, for describing one of the patient's conditions—tublointerstitial nephritis as well as a potential treatment with a steroidal treatment. However, the patient in the article is an adult.
12b) is dismissed as this article focuses on the in vitro testing of kidney cells.
12c) helpful, as this article studied the treatment options for renal allografts (transplants).
12d) is dismissed as the article focused on a patient with lupus erythematosus, a fatal condition in this case.
16a) helpful, as the article discusses the positive use of Cycosporin A in the treatment of tublointerstitial nephritis.
16b) helpful, as the article discusses the use of imuran against malignant lymphomas.
This pared down list of article references were returned to the user with the reviewers comments. The articles that align with the patient symptoms were grouped and listed first. The remaining articles were attached for completeness. The article references, not the abstracts nor the full article, were returned to the inquirer.

TABLE 4
Reference Citations

| | |
|---|---|
| Ref. | Citation |

Immunological 2 × 2 matrix results: “autoimmune disease” and “unknown
immunodeficiency”

| | |
|---|---|
| a | Nat Genet, 35(4): 349–56 2003 |
| b | Leuk Lymphoma, 44(3): 383–9 2003 |
| c | Curr Allergy Asthma Rep, 1(5): 421–9 2001 |
| d | J Immunol, 167(12): 6724–30 2001 |
| e | Scand J Immunol, 51(2): 111–22 2000 |
| f | Arch Immunol Ther Exp (Warsz), 47(5): 289–97 1999 |
| g | Folia Microbiol (Praha), 43(5): 545–50 1998 |
| h | Mt Sinai J Med, 64(6): 406–8 1997 |
| i | Microbiol Immunol, 41(3): 221–7 1997 |
| j | Adv Pediatr Infect Dis, 10(): 369–90 1995 |
| k | Br J Ophthalmol, 68(3): 182–7 1984 |
| l | Pediatr Allergy Immunol. 2004 Jun; 15(3): 281–3. |

Immunological 2 × 2 matrix results: “autoimmune disease” and “lymphopenia”

| | |
|---|---|
| a | Accumulation of irreversible disability in multiple sclerosis: From |
| | epidemiology to treatment..Author/s: Confavreux C Vukusic S |
| | Year: 2005; Source: Clin Neurol Neurosurg |
| b | Volume: 63 Issue: 1 Page: 50–8 Year: 2006, Source: Scand J Immunol |
| c | Volume: 9 Page: 74–94 Year: 2006; Source: Curr Dir Autoimmun |
| d | Volume: 11 Issue: 11 Page: 1238–43 Year: 2005, Source: Nat Med |
| e | Volume: 106 Issue: 10 Page: 3483–9 Year: 2005, Source: Blood |
| f | Volume: 25 Issue: 1 Page: 46–56 Year: 2005; Source: J Autoimmun |
| g | Volume: 78 Issue: 3 Page: 575–84 Year: 2005; Source: J Leukoc Biol |
| h | Volume: 24 Issue: 1–2 Page: 93–110; Source: Int Rev Immunol |
| i | Volume: 201 Issue: 5 Page: 723–35 Year: 2005; Source: J Exp Med |
| j | Volume: 22 Issue: 6 Page: 713–21; Source: Clin Exp Rheumatol |
| k | Volume: 174 Issue: 2 Page: 680–7 Year: 2005; Source: J Immunol |
| l | Volume: 34 Issue: 9 Page: 2396–406 Year: 2004; Source: Eur J Immunol |
| m | Volume: 101 Issue: 28 Page: 10398–403 Year: 2004; Source: Proc Natl Acad Sci |
| | USA |
| n | Volume: 117 Issue: 2 Page: 265–77 Year: 2004; Source: Cell |
| o | Volume: 81 Page: 331–71 Year: 2003; Source: Adv Immunol |
| p | Volume: 21 Issue: 4 Page: 315–24 Year: 2003; Source: J Autoimmun |
| q | Volume: 170 Issue: 7 Page: 3915–25 Year: 2003; Source: J Immunol |
| r | Volume: 170 Issue: 4 Page: 1667–74 Year: 2003; Source: J Immunol |
| s | Volume: 80 Issue: 6 Page: 567–73 Year: 2002; Source: Immunol Cell Biol |
| t | Volume: 169 Issue: 9 Page: 4850–60 Year: 2002; Source: J Immunol |
| u | Volume: 168 Issue: 12 Page: 5979–83 Year: 2002; Source: J Immunol |
| v | Volume: 3 Issue: 3 Page: 76–82 Year: 2002; Source: JOP |
| w | Volume: 34 Issue: 2 Page: 147–54 Year: 2001; Source: Autoimmunity |
| x | Volume: 167 Issue: 12 Page: 6724–30 Year: 2001; Source: J Immunol |
| y | Volume: 126 Issue: 3 Page: 540–4 Year: 2001; Source: Clin Exp Immunol |
| z | Volume: 44 Issue: 9 Page: 1998–2008 Year: 2001; Source: Arthritis Rheum |
| aa | Volume: 12 Issue: 11 Page: 1483–97 Year: 2000; Source: Int Immunol |
| bb | Volume: 23 Issue: 2 Page: 124–8 Year: 2000; Source: Nihon Rinsho |
| | Meneki Gakkai Kaishi |
| cc | Volume: 39 Issue: 2 Page: 142–7 Year: 2000; Source: Rheumatology |
| | (Oxford) |
| dd | Volume: 42 Issue: 9 Page: 1833–42 Year: 1999; Source: Arthritis Rheum |
| ee | volume: 104 Issue: 3 Pt 1 Page: 653–8 Year: 1999; Source: J Allergy Clin |
| | Immunol |
| ff | Volume: 103 Issue: 5 Pt 1 Page: 865–72 Year: 1999; Source: J Allergy Clin |
| | Immunol |
| gg | Volume: 135 Issue: 1 Page: 54–6 Year: 1999; Source: Arch Dermatol |
| hh | Volume: 197 Issue: 2 Page: 137–40 Year: 1998; source: Dermatology |
| ii | Volume: 157 Issue: 2 Page: 201–5 Year: 1998; Source: J Neurol Sci |
| jj | Volume: 25 Issue: 1 Page: 238–44 Year: 1995; Source: Eur J Immunol |
| kk | Volume: 54 Issue: 3 Page: 236–44 Year: 1993; Source: J Leukoc Biol |
| ll | Volume: 124 Issue: 2 Page: 152–8 Year: 1991; Source: Acta Endocrinol |
| | (Copenh) |
| mm | Volume: 33 Issue: 4 Page: 436–43 Year: 1989; Source: Am J Med Genet |
| nn | Volume: 1 Issue: 5 Page: 389–97 Year: 1988; Source: J Autoimmun |
| oo | Volume: 140 Issue: 11 Page: 1194–6 Year: 1986; Source: Am J Dis Child |
| pp | Volume: 136 Issue: 3 Page: 856–9 Year: 1986; Source: J Immunol |
| qq | Volume: 1 Issue: 3 Page: 224–32 Year: 1983; Source: Diagn Immunol |
| rr | Volume: 1 Issue: 1 Page: 35–42 Year: 1982; Source: J Pediatr Gastroenterol |
| | Nutr |
| ss | Volume: 11 Issue: 1 Page: 173–84 Year: 1975; Source: Birth Defects Orig |
| | Artic Ser |
| tt | Intern Med. 2005 Dec; 44(12): 1312–5 |
| uu | Rheum Dis Clin North Am. 2002 Aug; 28(3): 561–77, vi–vii. |

Immunological 2 × 2 matrix results: “autoimmune disease” and “prednisone”

too many non-specific articles

Immunological 2 × 2 matrix results: “unknown immunodeficiency” and
“lymphopenia”

no specific hits

Immunological 2 × 2 matrix results: “unknown immunodeficiency” and “prednisone”

| | |
|---|---|
| a | Volume: 19 Issue: 1 Page: 56–9; Source: Pediatr Dermatol |
| b | Volume: 91 Issue: 1 Page: 155–63 Year: 2001; Source: Cancer |
| c | Volume: 10 Issue: 2 Page: 189–95 Year: 1999; Source: Ann Oncol |
| d | Volume: 25 Issue: 3 Page: 254–6 Year: 1998; Source: Can J Neurol Sci |
| e | Volume: 12 Issue: 3 Page: 231–3 Year: 1998; Source: Pediatr Nephrol |
| f | Volume: 195 Issue: 4 Page: 344–8 Year: 1997; Source: Dermatology |
| g | Volume: 121 Issue: 8 Page: 853–8 Year: 1997; Source: Arch Pathol Lab |
| | Med |
| h | Volume: 192 Issue: 2 Page: 164–6 Year: 1996; Source: Dermatology |
| i | Volume: 13 Issue: 6 Page: 759–61; Source: Clin Lxp Rheumatol |
| j | Volume: 102 Issue: 11 Page: 1255–9 Year: 1992; Source: Laryngoscope |
| k | Volume: 34 Issue: 1 Page: 92–6 Year: 1989; Source: Dig Dis Sci |
| l | Volume: 260 Issue: 20 Page: 3045–8 Year: 1988; Source: JAMA |
| m | Volume: 5 Issue: 2 Page: 1 19–23 Year: 1987; Source: Asian Pac J Allergy |
| | Immunol |
| n | Volume: 42 Issue: 2 Page: 447–52 Year: 1978; Source: Cancer |
| o | Haematologia (Budap). 2002; 32(4): 535–41. |

Immunological 2 × 2 matrix results: “lymphopenia” and “prednisone”

too numerous to list

Nephrological 2 × 2 matrix results: “eosinophilic folliculitis” and “peripheral
eosinophilia”

| | |
|---|---|
| a | Am J Hematol. 2004 Jul; 76(3): 295–6. |
| b | Am J Med. 1997 May; 102(5): 449–53. |
| c | Arch Dermatol. 1991 Feb; 127(2): 206–9. |

Nephrological 2 × 2 matrix results: “eosinophilic folliculitis” and “renal tublar
acidosis”

not enough hits.

Nephrological 2 × 2 matrix results: “eosinophilic folliculitis” and “autoimmune
interstitial nephritis”

not enough hits.

Nephrological 2 × 2 matrix results: “eosinophilic folliculitis” and “imuran”

| | |
|---|---|
| a | Arch Dermatol. 1994 Mar; 130(3): 343–7. |

Nephrological 2 × 2 matrix results: “peripheral eosinophila” and “renal tubular
acidosis”

not enough hits.

Nephrological 2 × 2 matrix results: “peripheral eosinophilia” and “autoimmune
interstitial nephritis”

| | |
|---|---|
| a | Am J Kidney Dis. 2001 Dec; 38(6): E33. |
| b | Am J Kidney Dis. 2000 Oct; 36(4): 735–44. |
| c | Kidney Int. 1999 Jun; 55(6): 2457–66. |
| d | J La State Med Soc. 1996 Sep; 148(9): 379–84. |

Nephrological 2 × 2 matrix results: “peripheral eosinophilia” and “imuran”

not enough hits.

Nephrological 2 × 2 matrix results: “renal tubular acidosis” and “autoimmune
interstitial nephritis”

not enough hits.

Nephrological 2 × 2 matrix results: “renal tubular acidosis” and “imuran”

not enough hits.

Nephrological 2 × 2 matrix results: “autoimmune interstitial nephritis” and “imuran”

| | |
|---|---|
| a | Immunol Lett, 15(4): 301–6 1987 |
| b | Clin Exp Immunol, 3(4): 305–12 1968 |

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for obtaining references that assist in identifying a disease or condition in an individual, wherein the reference has been described or made part of a database having medical data, the method comprises:
a. obtaining or identifying two or more symptoms, conditions, procedures, medication or any combination thereof, experienced or used by the individual;
b. transcribing said symptoms, conditions, procedures, medication or combination thereof into standardized medical codes to thereby obtain transcribed codes;
c. categorizing transcribed codes by medical specialty;
d. searching at least one medical literature database using one of the transcribed codes to thereby obtain code-specific results, and repeating this step for each of the transcribed codes;
e. cross-referencing the code-specific results for each transcribed code with code-specific results of all other transcribed codes in the same categorized medical specialty to obtain common references, and repeating this step for each specialty for which the codes have been categorized; and
f. studying said references to determine references applicable to or those that assist in identifying said disease or condition.

2. The method of claim 1, further includes studying a medical chart for the individual to obtain or identify two or more symptoms, conditions, procedures, medication or any combination thereof, exhibited or used by the individual.

3. The method of claim 1, wherein the standardized medical codes are International Classification of Diseases (ICD) codes, Current Procedural Terminology (CPT) codes, National Drug Codes (NDC) codes, or combination thereof.

4. The method of claim 1, further includes obtaining one or more demographics of the individual.

5. The method of claim 4, further includes filtering out references having inconsistent demographics with the demographics of the individual.

6. The method of claim 5, wherein obtaining one or more demographics of the individual includes obtaining age, sex, race, species, geographic location or combination thereof.

7. The method of claim 6, wherein filtering out results having inconsistent demographics involves excluding results that describe a demographic that is different than the demographic obtained from the individual.

8. The method of claim 1, wherein cross-referencing the code-specific results with code-specific consistent results of all other transcribed codes in the same categorized medical specialty includes making a two dimensional graph or representation that compares results for each transcribed code.

9. A method for obtaining references that assist in identifying a disease in an individual, wherein the references have been described or made part of a database having medical data, the method comprises:
a. obtaining or identifying two or more symptoms, conditions, procedures, medication or any combination thereof, experienced or used by the individual;
b. transcribing said symptoms, conditions, procedures, medication or any combination thereof into standardized medical codes to thereby obtain transcribed codes;
c. categorizing transcribed codes by medical specialty;
d. cross-referencing each transcribed code with all other transcribed codes in the same categorized medical specialty to thereby obtain cross-referenced code sets or pairs;
e. using the cross-referenced code sets, searching at least one medical literature database to thereby obtain cross-referenced, code-specific results, and repeating this step for each set or pairs of cross-referenced code; and
f. studying said results to determine references applicable to or those that assist in identifying said disease or condition.

10. The method of claim 9, further including identifying an orphan disease that has been described and made part of a database having medical information.

11. The method of claim 9, further including studying a medical chart for the individual to obtain or identify symptoms, conditions, procedures, medication or any combination thereof experienced or used by the individual includes.

12. The method of claim 9, wherein the standardized medical codes are International Classification of Diseases (ICD) codes, Current Procedural Terminology (CPT) codes, National Drug Codes (NDC) codes, or combination thereof.

13. The method of claim 9, further includes obtaining one or more demographics of the individual.

14. The method of claim 13, further includes filtering out results having inconsistent demographics with the demographics of the individual.

15. The method of claim 14, wherein obtaining one or more demographics of the individual includes obtaining age, sex, race, species, geographic location or combination thereof.

16. The method of claim 15, wherein filtering out results having inconsistent demographics involves excluding results that describe a demographic that is different than the demographic obtained from the individual.

17. The method of claim 16, wherein cross-referencing the code-specific results with code-specific results of all other transcribed codes in the same categorized medical specialty includes making a two dimensional graph or representation that compares results for each transcribed code.

18. A method for obtaining references that assist in identifying a disease in an individual wherein the reference has been described or made part of a database having medical data, the method comprises:
a. obtaining or identifying two or more symptoms, conditions, procedures, medication or any combination thereof, experienced or used by the individual;
b. obtaining one or more demographics of the individual;
c. transcribing said symptoms, conditions, procedures, medication or any combination thereof into standardized medical codes to thereby obtain transcribed codes;
d. categorizing transcribed codes by medical specialty;
e. searching at least one medical literature database using one of the transcribed codes to thereby obtain code-specific results, and repeating this step for each of the transcribed codes;
f. filtering out results having inconsistent demographics to thereby obtain code-specific, demographic consistent results;
g. cross-referencing the code-specific, demographic consistent results for each transcribed code with code-specific, demographic consistent results of all other transcribed codes in the same categorized medical specialty to obtain common references, and repeating this step for each specialty for which the codes have been categorized; and
h. studying said references to determine references applicable to or those that assist in identifying said disease condition.

19. The method of claim 18. further including providing an output of the results and applicable references for identifying the said disease.

20. The method of claim 19, wherein the output groups the references according to relevance.

21. The method of claim 20, wherein the transcribed code represents a symptoms, conditions, procedures, medication or any combination experienced or used by the individual.

22. A method for obtaining references that assist in identifying a disease or condition in an individual wherein the reference has been described or made part of a database having medical data, the method comprises:
a. identifying two or more symptoms, conditions, procedures, medication or any combination thereof, experienced or used by the individual;
b. transcribing said symptoms, conditions, procedures, medication or any combination thereof into standardized medical codes to thereby obtain transcribed codes;
c. categorizing transcribed codes by medical specialty;
d. searching at least one medical literature database using one of the transcribed codes to thereby obtain code-specific results, and repeating this step for each of the transcribed codes;
e. cross-referencing the code-specific results for each transcribed code with code-specific consistent results of all other transcribed codes in the same categorized medical specialty to obtain common references, and repeating this step for each specialty for which the codes have been categorized; and
f. providing an output that groups each common reference by the number of occurrences of transcribed codes, combinations of transcribed codes, or other key terms present in said reference.

23. The method of claim 22, further including studying said references to determine references applicable for identifying said disease.

24. In a computer system, a method for obtaining references that assist in a disease in an individual wherein the reference has been described or made part of a database having medical data, and wherein two or more symptoms, conditions, procedures, medication or any combination thereof experienced or used by the individual have been transcribed into standardized medical codes to thereby obtain transcribed codes and categorized by medical specialty; the method comprises:
a. searching at least one medical literature database using one of the transcribed codes to thereby obtain code-specific results, and repeating this step for each of the transcribed codes;
b. cross-referencing the code-specific results for each transcribed code with code-specific consistent results of all other transcribed codes in the same categorized medical specialty to obtain common references, and repeating this step for each specialty for which the codes have been categorized; and
c. providing an output that groups each common reference by the number of occurrences of transcribed codes present in said reference.

25. The method of claim 24, further includes obtaining one or more demographics of the individual.

26. The method of claim 25, further includes filtering out results having inconsistent demographics with the demographics of the individual.

27. A computer apparatus for obtaining references that assist in identifying a disease in an individual wherein the reference has been described or made part of a database having medical data, the apparatus comprises:
a. a source of transcribed codes that comprises standardized medical codes translated from two or more symptoms, conditions, procedures, medication or any combination thereof experienced or used by the individual, and the medical specialty associated with said codes;
b. a processor routine coupled to receive transcribed codes from the source, wherein the processor routine comprises the steps of:
i. categorizing transcribed codes by medical specialty;
ii. searching at least one medical literature database using one of the transcribed codes to thereby obtain code-specific results, and repeating this step for each of the transcribed codes;
iii. cross-referencing the code-specific results for each transcribed code with code-specific results of all other transcribed codes in the same categorized medical specialty to obtain common references, and repeating this step for each specialty for which the codes have been categorized; and
c. an output device, coupled to the processor routine, for indicating common reference grouped by the number of occurrences of transcribed codes present in said reference.

28. The computer apparatus of claim 27, further comprising a filter, coupled to the source, for filtering out any results having inconsistent demographics with the demographics of the individual.

29. The computer apparatus of claim 28, wherein demographics to be filtered out include age, sex, race, species, geographic location or any combination thereof.

30. A computer system for obtaining references that assist in identifying a disease or condition in an individual wherein the reference has been described or made part of a database having medical data, the system comprises:
a. a source of transcribed codes that comprises standardized medical codes translated from two or more symptoms, conditions, procedures, medication or any combination thereof experienced or used by the individual;
b. a processor routine coupled to receive transcribed codes from the source, wherein the processor routine comprises the steps of:
i. categorizing transcribed codes by medical specialty;
ii. searching at least one medical literature database using one of the transcribed codes to thereby obtain code-specific results, and repeating this step for each of the transcribed codes;
iii. cross-referencing the code-specific results for each transcribed code with code-specific results of all other transcribed codes in the same categorized medical specialty to obtain common references, and repeating this step for each specialty for which the codes have been categorized; and
c. an output device, coupled to the processor routine, for indicating common reference grouped by the number of occurrences of transcribed codes present in said reference.
